(19) United States
(12) Patent Application Publication
(10) Pub. No.: US 2023/0303780 A1
(43) Pub. Date: Sep. 28, 2023

(54) PROCESS FOR PREPARING SPHERICAL POLYMERIC PARTICLES FOR COSMETIC APPLICATION

(71) Applicant: RHODIA BRASIL S.A., São Paulo City (BR)

(72) Inventors: Tarcis Cordeiro Bastos, São Paulo City (BR); Edson Rodrigues Leme, São Paulo City (BR); Guilherme Lopes Do Lago, São Paulo City (BR)

(73) Assignee: RHODIA BRASIL S.A., São Paulo City (BR)

(21) Appl. No.: 18/041,503

(22) PCT Filed: Jul. 8, 2021
(86) PCT No.: PCT/IB2021/056111

§ 371 Date: Feb. 13, 2023

(30) Foreign Application Priority Data
Aug. 12, 2020 (WO) PCT/IB2020/057561

(51) Int. Cl. C08J 3/09; C08J 3/215; A61K 8/85; A61Q 19/08; A61K 8/02; A61K 8/29; A61K 8/23; A61K 8/25

(52) U.S. Cl. CPC C08J 3/095; C08J 3/215; A61K 8/85; A61Q 19/08; A61K 8/025; A61K 8/29; A61K 8/23; A61K 8/25; C08J 2367/02; C08K 2003/2241

(57) ABSTRACT

A process for preparing spherical polymeric particles of polymers containing at least two fillers, dispersed in the polymer matrix wherein up to 50% of the weight of the particles is composed by fillers. The process comprises a melt-blending of a polymeric matrix containing the at least two fillers with a continuous phase that is not miscible with the polymeric matrix and an agent to form an emulsion. This emulsion is then extruded, cooled and a solvent of the continuous phase is added to recover the spherical particles. The fillers can provide different properties to the spherical particles which can be used, for example, for cosmetic applications, specifically for preventing and/or reducing the signs of skin ageing.

### FIELD OF THE INVENTION

This invention relates to a process for preparing spherical polymeric particles containing at least two mineral fillers dispersed in the polymer matrix wherein up to 50% of the weight of the particles is composed by fillers. The process comprises a melt-blending of a polymeric matrix containing at least two fillers with a continuous phase compound and an agent to form an emulsion, cooling, providing the solubilization of continuous phase and recovering the spherical particles. The present invention also relates to the use of the spherical polymeric particles for cosmetic applications, specifically for preventing and/or reducing the signs of skin ageing.

### BACKGROUND OF THE INVENTION

The present invention relates to a process for producing spherical polymeric particles containing fillers.

The subject of the present invention is also the use of such a spherical polymeric particle in the cosmetic field, having biostimulatory properties for preventing or reducing the signs of skin ageing.

Spherical particles as an additive carrier can be used in a wide variety of applications including cosmetics, paints, coatings, selective laser sintering, adhesives, waxes, lubricants and paper.

Polymers in the powder form, in particular in the form of spherical particles with a controlled diameter generally of less than 1 mm, preferably of less than 100 μm, can improve some characteristics in several applications. These spherical particles can be used as an additive in paints, for example, in paints for coating the floor of sports halls, which must have nonslip properties, or can also be introduced into cosmetic products such as sunscreen, creams for caring the body or face, and make-up-removing products. They are also used in the field of inks and papers.

Different technologies have been used to control the spherical shape of polymeric particles.

Some technologies have been used for preparing spherical particles of polymers with diameter less than 1 micron. For example, it is known to obtain polymer powders, such as polyamide powders, by anionic polymerization of lactams in solution. However, this technology is limited concerning the nature of the polymer and the control of particle size is difficult due to the high reactivity of the anionic monomers.

Published PCT application no. WO2005/000456 discloses the technology used for production of spherical particles made of polyamide. Spherical particles with size less than 100 microns were obtained using emulsion polymerization. However, the control of particle size is limited.

United States Patent Application Publications Nos. 2010/009189 and 2009/072424 to Herve et al disclose a process where it is possible to obtain spherical particles with controlled size for polyamide. The processes use the high internal phase emulsion (HIPE) principle in which the soluble material is the continuous phase. The particles are obtained from a molten blend between polymer particles and a soluble polymer and, the application of a mixing energy in an extruder allows the formation of discrete particles of the thermoplastic material dispersed in the continuous phase formed by the soluble polymer. The melt blend is cooled and the particles are separated by solubilization of the continuous phase. However, those processes are well implemented for polyamides using a proper self-developed additive having the same polyamide nature in hyperbranched form, an expensive and laborious obtained additive. Once the nature of polymer changes, many drawbacks are faced in order to change the continuous phase and still obtain spherical particles with controlled size.

United States Patent Application Publication No. 2015/0147364 discloses a cosmetic composition containing spherical particles of polyamide within which are dispersed mineral fillers. The process of preparation of polyamide particles described uses HIPE principle and a proper self-developed additive having the same polyamide nature in hyperbranched form in its struture, an expensive and laborious obtained additive.

The ability to get high volume fraction of dispersed droplets in a low volume fraction of continuous phase have become the HIPEs technique attractiveness for use in a wide range of areas for example as templates for porous materials used in various applications, such as organic semiconductors, filter membranes, scaffolds for tissue engineering, food products and drug delivery systems. In this case, the dispersed droplets are constituted by a soluble polymer that is removed after the co-extrusion process, resulting in a porous material.

### SUMMARY OF THE INVENTION

Despite advances in technologies for the production of polymeric particles in a controlled way, there is a need to provide a process able to control the shape and size of said particles, which can be applied in different kind of polymers and, in the same time, allow its particles to have additives, as fillers, with good dispersibility at the polymeric matrix.

Pursuing its research in this field, the Applicant has now discovered an original process for preparing fine spherical polymeric particles, applicable in different types of polymers, with controlled spherical shape in a way that also ensures that the filler will remain dispersed in the polymeric matrix.

A first object of the present invention is, therefore, to provide a process to produce spherical polymeric particles able to contain up to 50% of at least two fillers dispersed in the polymer matrix.

The process of the invention makes possible to manufacture spherical particles from any polymeric thermoplastic material, both synthetic and biodegradable ones, the latter having the advantage of being sustainable.

Accordingly, the present invention provides a process for preparing spherical particles comprising a thermoplastic polymer matrix M comprising at least two fillers F, dispersed in the thermoplastic polymeric matrix M, which comprises the following steps:

A—melt-blending a mixture comprising:
a) at least one thermoplastic polymer matrix M comprising up to 50% of at least two fillers F dispersed therein;
b) at least one compound P, different from the at least one thermoplastic polymer M, not miscible with the at least one thermoplastic polymer matrix M and selected in the group consisting in polyglycols, polysaccharides, polyolefins, polyvinyl alcohols, silicones, waxes, and mixtures thereof, and
c) at least one agent C which is an amphiphilic compound having a first part of its structure that can react chemically or physically with the thermoplastic polymer matrix M and a second part of its structure that can react chemically or physically with the compound P, and in which the first part of its structure does not contain a polymer chain identical to the thermoplastic polymer matrix M;
thus forming an emulsion containing a continuous phase of compound P and agent C and droplets of thermoplastic polymer matrix M and filler F;
B—cooling the melt blend obtained at step A at a temperature below the softening temperature of the blend,
C—putting the cooled blend into a solvent wherein compound P and agent C are soluble to provide the solubilization of compound P and agent C,
D—recovering spherical particles comprising the thermoplastic polymer matrix M and the at least two fillers F dispersed therein.

By virtue of such process, it is possible to obtain spherical polymeric particles according to the invention, which contain at least two fillers dispersed in the polymeric matrix.

Accordingly, the present invention provides a process, which is carried out by using the HIPEs principle.

The ability to create HIPEs with controlled small droplets and keep the additives inside the droplets, as a filler, is a significant challenge.

The invention is based on the discovery that the inclusion of a proper agent C during the emulsion formed by a blend between a disperse thermoplastic polymer matrix M, the fillers F and a continuous phase, compound P, has not only an effect on stabilizing the emulsion, but also guarantees the proper dispersion of the fillers F in the thermoplastic polymeric matrix M.

According to the invention, the spherical particles of different types of polymers are provided by using the same agent C.

Indeed, the Applicant has discovered, totally unexpectedly, that the agent C, which does not contain part of its structure a polymer chain identical to the thermoplastic polymer matrix M; is the key to stabilize the spherical shape of the polymeric particles and also to ensure the fillers F will remain dispersed in the thermoplastic polymeric matrix M during the co-extrusion process.

Another challenge is to ensure that the affinity between the compound P and thermoplastic polymeric matrix M be enough to enable the emulsion formation with an ideal viscosity difference between the phases that will contribute to the stress share.

By “affinity” is intended to mean a similarity of characteristics suggesting a possible chemical or physical reaction/interaction between different compounds or mixture of compounds.

The viscosity of a fluid, like an emulsion, is a measure of its resistance to deformation at a given rate. Generally, the process of emulsification and rupture of isolated droplets depends on the viscosity ratio between internal and external phases and the mechanical shear stress. Small drop sizes are favoured by increasing of external phase viscosity. On the other side, viscosity increase demands more energy to form the emulsion.

Thus, the viscosity of the external phase can play an important role in both the emulsification process and the viscosity of the final emulsion. During emulsification, a higher viscosity will produce a lower final drop size. However, there is a critical external polymer viscosity/internal polymer viscosity ratio in which droplet size and shape are difficult to obtain.

According to the present invention, the use of a proper agent C is key through its effect on interfacial properties and continuous phase rheology, which is related to time-dependent deformation of bodies under the influence of applied stresses.

A second object of the present invention is the use of the functional biostimulatory effect of the spherical polymeric particles obtained, which can be provided by different fillers, particularly in the cosmetic field, for preventing and/or reducing the signs of skin aging.

In a manner known per se, the term “signs of skin aging” denotes the marks present on the skin resulting from aging phenomena, which modify its visual appearance and are generally considered to be unattractive, such as, in particular, wrinkles and age spots.

### BRIEF DESCRIPTION OF THE DRAWINGS

The advantages described above are clearer to those skilled in the art from the figures:

FIG. 1 shows the Scanning Electron Microscopy (SEM) image (SEI, 15 kV, 230×) obtained for polyamide 6.6 spherical particles produced by the co-extrusion process of the invention.

FIG. 2 shows the Scanning Electron Microscopy (SEM) image (SEI, 15 kV, 450×) obtained for polyhydroxybutyrate (PHB) spherical particles produced by the co-extrusion process of the invention.

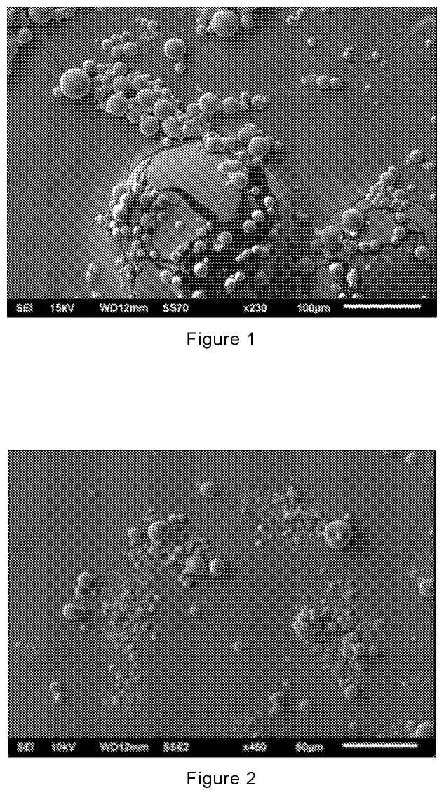

### DEFINITIONS

Throughout the description, including the claims, all process terms should be understood as being synonymous with the term method.

As ASTM definition, the term “biodegradable polymers” refers to the degradation from the action of naturally-occurring microorganisms such as bacteria, fungi and algae. As a result, biodegradable materials degrade into biomass, carbon dioxide and methane, which have special properties like non-toxicity, biocompatibility and biodegradability. When biodegradability takes place in marine environment, the polymers are marine biodegradable polymers.

As used herein, the term “biostimulatory effect” refers to biological effects on skin integrity, enhancing its appearance and relieve skin conditions.

As used herein, the term “soluble” refers to the 99% of recovery of the compound P and the agent C at a temperature of 25° C.

“Amphiphilic” is a term describing a chemical compound possessing both hydrophilic and hydrophobic properties. Such a compound is called amphiphilic or amphipathic.

An “emulsion” is a suspension made of a first liquid in a phase made of a second liquid with which the first liquid is not miscible with the second liquid. A discontinuous phase within a continuous phase is then obtained.

### DETAILED DESCRIPTION OF THE PREFERRED EMBODIMENTS

The present invention is based on a process for preparing spherical particles comprising a thermoplastic polymer matrix M and at least two mineral fillers F dispersed wherein.

In one embodiment, the thermoplastic polymer matrix M can be chosen in particular from the group comprising: polyesters, polyolefins, polymers based on a cellulose ester, such as cellulose acetate, cellulose propionate and polymers of the same family, acrylic polymers and copolymers, polyamides such as polyhexamethylene adipamide (PA66), polycaprolactam (PA6), polyamide 5.6, PA6.10, PA10.10 and PA12, copolymers in any proportions of these polymers, and blends between any of these polymers.

According to one preferential embodiment, the thermoplastic polymer matrix M consists of polyamide, preferably chosen from polyamide 6, polyamide 66, polyamide 56 and copolymers of polyamide 6/polyamide 66, polyamide 6/polyamide 56 and polyamide66/polyamide 56 in any proportions.

In another embodiment, the thermoplastic polymer matrix M consists of a marine biodegradable polymer which denotes any polymer with intrinsic character of biodegradability as for example:
polyhydroxybutyrate (PHB),
polyhydroxybutyrate-co-valerate (PHBV).
polylactic acid (PLA),
polylactic-co-glycolic acid (PLGA),
polyhydroxyalkanoates (PHAs),
thermoplastic starches (TPS),
poly(butylene Succinate) (PBS),
poly(butylene Succinate adipate) (PBSA),
polybutylene adipate (PBA)
polybutylene adipate terephthalate (PBAT) or blend like Polylactic acid (PLA)/polycaprolactone (PCL), and
a thermoplastic polymer made with additives that provide a biodegradable character.

As examples of additives that provide a biodegradable character for a thermoplastic polymer, mention can be made, for example, of commercially available additives under the names BioSphere® 201 and Ecopure® CNY-EP-04C-NY.

According to one preferential embodiment, the thermoplastic polymer matrix M consists of polyhydroxyalkanoates (PHAs), preferably chosen from polyhydroxybutyrate (PHB) and polyhydroxybutyrate-co-valerate (PHBV).

According to the invention, the thermoplastic polymer matrix M containing up to 50% of dispersed fillers F is used in the solid form, particularly as granules.

In general, the granules previously extruded containing the polymer and at least two fillers F, are prepared before the melt blending.

According to one embodiment, the said granules are present in the melt blend emulsion in an amount of less than 80 wt.% and more than 15 wt.%, based on the total weight of the emulsion, preferable less than 50 wt. % and more than 20 wt. %.

Fillers F:

According to the invention, the fillers F are dispersed in the thermoplastic polymer matrix M. The term “dispersed” is intended to mean that the fillers F are mostly incorporated inside the thermoplastic polymeric matrix M and/or inside the spherical particles. In particular, the fillers are trapped in the polymer matrix and/or particles. They are not therefore mineral fillers deposited on the polymer, for example in the form of a coating at the surface of the polymer.

In one embodiment, said fillers F can be incorporated in the thermoplastic polymer matrix M, for example, by an extrusion process and then granulated, or during the polymerization process, advantageously at the end of polymerization. It is also possible to introduce the fillers F into the polymer in the molten state.

In one embodiment, the process of the present invention results in spherical polymer particles having at least two fillers F dispersed in the thermoplastic polymer matrix M, which can advantageously promote biostimulatory effect.

According to the invention, biostimulatory effect can be provided by organic or inorganic fillers, which have the capability for absorption/emission of radiation in the infrared region, incorporated into a polymeric substrate. Preferably, mineral fillers F with far infrared emitting (FIR) properties in region ranging from 3 to 20 μm, and even more preferably from 3 to 15 μm.

The infrared radiation absorption spectrum can be determined by any method known to those skilled in the art. One possible method is the use of a Bruker Equinox 55 instrument, with a resolution of 4 cm<-1>. In this case, the spectrum obtained is in ATR (“Attenuated Total Reflectance”) form, using a ZnSe crystal.

The mineral fillers F usable according to the invention can be chosen from a combination of following groups: oxide groups, sulfate groups, carbonate groups, silicate groups and the phosphate groups.

Preferably, the oxides are chosen from titanium dioxide, silicon dioxide and magnesium oxide.

The sulfates can advantageously be chosen from barium sulfate, calcium sulphate and strontium sulphate.

Preferably, the carbonates are chosen from calcium carbonate or sodium carbonate.

The phosphates can advantageously be chosen from zirconium phosphates, calcium phosphate, hydroxyapatite, apatite, magnesium phosphate, sodium phosphate, potassium phosphate and other possible phosphates.

Preferably, the silicates are chosen from actinolite, micas, tourmaline, serpentine, kaolin, montmorillonite, zeolite and other aluminum silicate, preferably tourmaline.

In one embodiment, at least one mineral filler F is a silicate, preferably selected in the group consisting of actinolite, micas, tourmaline, serpentine, kaolin, montmorillonite, zeolite and other aluminum silicate and mixtures thereof, more preferably tourmaline.

In another embodiment, the mineral fillers F are selected preferably from the group consisting of oxides, sulphates and silicates, more preferably being titanium dioxide, barium sulphate and tourmaline.

According to one embodiment of the present invention, the weight proportion of fillers F relative to the total weight of the spherical particle is greater than or equal to 1%, preferably greater than, or equal to 5% and even more preferably greater than or equal to 15%.

In another embodiment, the weight proportion of fillers F relative to the total weight of the spherical particle is less than or equal to 50%, preferably less than or equal to 35% and even more preferably less than or equal to 30%.

Compound P

According to the process of the present invention, the compound P is different from the at least one thermoplastic polymer M and not miscible with the at least one thermoplastic polymer matrix M.

In one embodiment, the compound P is selected in the group consisting in polyglycols, polysaccharides, polyolefins, polyvinyl alcohols, silicones, waxes, and mixtures thereof.

Preferably, the polyglycol chosen was polyethylene glycol (PEG).

Advantageously, the compound P is selected in the group consisting of polyoxyethylenes (POE) and polyalkylene glycols (PAG), preferably polyethylene glycols (PEG).

According to one embodiment, the particular polymer used as compound P of the present invention is a polyethylene glycol (PEG) with a molecular weight ranging from 1500 to 60000 g/mol, preferably from 6000 to 35000 g/mol.

In another embodiment, the proportion by weight of the compound P by weight of the blend of the invention from 15 to 80 wt. % of compound P preferably being a PEG, preferably from 40 to 70 wt. %;

Agent C:

According to the process of the present invention, the agent C, is an amphiphilic compound having a first part of its structure that can react chemically or physically with the thermoplastic polymer matrix M and a second part of its structure that can react chemically or physically with the compound P, and in which the first part of its structure does not contain a polymer chain identical to the thermoplastic polymer matrix M.

As examples for the present invention, the agent C is ethoxylated/propoxylated block copolymer,

In a preferred embodiment, the agent C ethoxylated (EO)/propoxylated (PO) block polymers (EO/PO) used by the present invention has appropriate HLB and molecular weight. These kind of polymers are amphiphilic molecules consisting of hydrophilic ethylene oxide (EO) and hydrophobic propylene oxide (PO) blocks. Thus, the amphiphilic character of molecules like EO/PO block copolymers can be characterized by the hydrophilic-lipophilic balance (HLB). Several experimental and numeric methods have been developed over the years to determine HLB numbers.

The appropriate EO/PO copolymers, with the desired molecular weight and HLB, allow the formation of stable HIPEs for emulsions containing a polyester as the dispersed phase (such as polyhydroxybutyrate—PHB, polyhydroxybutyrate-co-valerate—PHBV or polyhydroxyalkanoates—PHAs) and continuous phase like polyethylene glycol (PEG). The EO block from the copolymer appears to be solubilized in the more polar polymer (compound P) while the PO block appears to be solubilized in the less polar phase, the dispersed thermoplastic polymeric matrix M.

An increase in EO ratio of the agent C implies an increase in HLB value, which directly impacts on the stability of the HIPE and, consequently, at the final spherical polymeric particles formation.

The lipophilic part of the agent C will interact more strongly with the thermoplastic polymeric matrix M, creating a kind of barrier that will bring two benefits. First, it will hinder the migration of the fillers F and second, it will reduce the interfacial tension, mitigating the deformation of the formed droplets.

However, the affinity of the fillers F in the compound P must be reduced towards thermoplastic polymer matrix M in order to ensure its maximum inclusion into the said matrix M.

The agents C advantageously selected for the process of the present invention are ethoxylated/propoxylated block copolymers (EO/PO) of appropriate HLB and molecular weight.

Particularly, the agent C of the present invention is an ethoxylated/propoxylated block polymer with a molecular weight ranging from 500 to 10000 g/mol, preferably from 3000 to 7000 g/mol.

In one embodiment, the agent C is an ethoxylated (EO)/propoxylated (PO) block polymer with a PO/EO ratio ranging from 2 to 10, preferably from 5 to 7.

According to another embodiment, the weight proportion of the agent C by weight of the blend of the invention is selected from 1 to 20 wt. %, preferably from 5 to 10 wt. %.

According to a preferred embodiment, the step A of melt-blending takes place at a temperature above 100° C. and below 300° C., preferably above 160° C. and below 270° C.

Particularly, the melt blend of the present invention is processed by extrusion in an extruder selected from endless screw mixers or stirrer mixers, preferably, the extruder is a twin-screw extruder or a multi-screw extruder.

Typically, the extrusion process of the present invention occurs with the rotation extruder at about 100 to 600 rpm, more specifically between 200 to 500 rpm.

The step B of cooling the melt blend obtained at step A is conducted by any appropriate means, most often at a temperature below the softening temperature of the blend. Mention can notably be made by air cooling or quenching in a liquid.

In a preferred embodiment, the blend where thermoplastic polymer matrix M is PHB or PA 6.6, compound P is PEG and agent C is ethoxylated/propoxylated (EO/PO) block copolymers the step B is conducted at a temperature in a range from 15 to 40° C.

The step C of the present invention is commonly conducted by immersing the cooled blend obtained at step B into a bath containing a solvent wherein compound P and agent C are soluble to provide the solubilization of compound P and agent C.

Alternatively, the cooling step B and the solubilization step C can be made by the same solvent.

It is highly recommended that the compound P and agent C have small solubility and high incompatibility with the thermoplastic polymer matrix M. In this way, the solubilization process of compound P and agent C can take place without loss of spherical polymeric particles, increasing the yield of the process.

Usually, the solvent used in step C is selected in the group consisting of water, methanol, ethanol, isopropanol and butanol, preferably water.

Such a solubilization according to step C allows to produce a dispersion of the particles which can be isolated for instance by filtration, separation by settling, centrifugation or atomization.

If necessary during the solubilization step C, it is possible to apply a mechanical force, such as rubbing, shearing, grinding, sonication or twisting.

The step D of the present invention is conducted by recovering spherical particles comprising the thermoplastic polymeric matrix M and the at least two fillers F dispersed therein.

Advantageously, the spherical particles are then dried after step D. The step of drying can, for example, take place in an equipment like an oven, and at a temperature range from 30 to 110° C.

In an advantageous embodiment, the process of the present invention comprises a melt blend mixture of step A comprising:

a) from 15 to 80 wt. % of thermoplastic polymer matrix M+F, preferably being PHB or PA 6.6, comprising three fillers F being titanium dioxide, barium sulphate and tourmaline, preferably from 20 to 50 wt. %, and;
b) from 15 to 80 wt. % of compound P preferably being a PEG, preferably from 40 to 70 wt. %;
c) from 1 to 20 wt. % of agent C being an ethoxylated/propoxylated block copolymer, preferably from 5 to 10 wt. %.

The process of the invention makes possible the preparation of polymeric particles of regular shape and size.

As used herein, the term “particle” refers to an individualized entity.

The particles of the present invention can be characterized by their bulk, which means they can be characterized from a large amount.

According to a first preferred embodiment of the invention, the particles of polymeric composition have a substantially spherical shape, according to Scanning Electron Microscopy (SEM), i.e. the particles have a shape similar to that of a sphere, which may be more or less regular, for example spheroids, and/or ellipsoids.

The particles of the present invention can be characterized by their particle size distribution D50 (in short “D50”), which is also known as the median diameter or the medium value of the particle size distribution, according to which 50% of the particles in the sample are larger and 50% of the particles in the sample are smaller. Particle Size Analysis can for example take place in a Malvern Mastersizer 3000 laser granulometer.

According to one embodiment, the spherical particles of the present invention present the average particle size D50 ranging from 5 μm to 60 μm, preferably from 10 μm to 40 μm.

Migrated Fillers F from Particle

The main advantage of the present invention is that the majority (more than 50%) fillers F are located inside the spherical particles, which means, fillers F are dispersed in the thermoplastic polymer matrix M.

The content of fillers F that have migrated from thermoplastic polymeric matrix M to the surface of the particles, for example, PHB and PA 6.6, can be estimated from the Particle Size Analysis data, using volume difference between particles of thermoplastic polymeric matrix M and particles of free fillers F. The inorganic fillers F have the anti-ageing effect and are expensive, thus, avoiding its loss during the extrusion mixing step allows to have an economic process.

In one embodiment, the migrated fillers F parameter found for the particles varies according to the amount of agent C added during the melt blend step, varying from 20 to 5000 mg/kg by adding agent C and reaching 140000 mg/kg without addition of the agent C.

In one embodiment, the migrated fillers F from particles is not more than 5000 mg/kg, preferably not more than 3000 mg/kg and even more preferably not more than 2260 mg/kg.

Spherical Shape Factor

By the process of the present invention it is possible to obtain spherical particles.

The spherical shape of the particles can be evidenced by Scanning Electron Microscopy (SEM), which can provide direct observation of microstructural features on a surface, at an interface and inside a bulk material. A scanning electron microscope (SEM) is a type of electron microscope that produces images of a sample by scanning the surface with a focused beam of electrons. The electrons interact with atoms in the sample, producing various signals that contain information about the surface topography and composition of the sample.

The procedure for assessing sphericity of the polymeric particles was carried out by Scanning Electron Microscopy (SEM) using the major and minor axis passing through the center of the particle and the ratio obtained reflects the spherical shape factor ratio.

In one embodiment, the spherical shape factor ratio of the present invention is selected from 0.5 to 1.0, preferably to 0.75 to 1.0.

Applications:

The spherical polymeric particles of the present invention can be used in various applications, notably for cosmetic compositions, preferably for preventing or reducing the signs of skin ageing.

Illustrating the invention are the following examples that are not to be considered as limiting the invention to their details.

### EXPERIMENTAL PART

The present invention will be illustrated by way of the following examples.

In the examples, the various abbreviations have the following meaning.

PHB: polyhydroxybutyrate polymer. The PHB is obtained by BIOMER under the name of BIOMER biopolyesters.

PHB+FIR: polyhydroxybutyrate polymer plus a far infrared absorbing/emitting filler F.

PA 6.6: polyamide 6.6 polymer. The PA 6.6 is produced by Solvay and commercially available under the name of Polyamide 6.6 Brilliant.

PA 6.6+FIR: polyamide 6.6 plus a far infrared absorbing/emitting filler F.

Fillers F were obtained from Venator and Microservice under the name of titanium dioxide, barium sulphate and tourmaline.

PEG: polyethylene glycol polymer. The PEG is obtained by Sigma-Aldrich under the name of polyethylene glycol.

PEG 6000, PEG 20000 and PEG 35000: polyethylene glycol polymer with molecular weight of 6000, 20000 and 35000 g/mol, respectively.

Agent C is ethoxylated/propoxylated block copolymer and is commercially available from Solvay under the name of Antarox L 101.

d(0.1)=10% of the total volume is represented by particles with diameter smaller than d (0.1).

d(0.5)=50% of the total volume is represented by particles with diameter smaller than d (0.5).

d(0.9)=90% of the total volume is represented by particles with diameter smaller than d (0.9).

The twin-screw extruder equipment: Co-rotating twin-screw Coupled to Thermo Scientific Torque Rheometer—model Polylab OS Rheodrive 7/HAAKE Rheomex OS Extruder PTW16, L/D 16 mm.

Particle Size Analysis was measured by Malvern Mastersizer 3000 laser granulometer.

Scanning electron microscopy was performed in a JEOL JSM-6610LV SEM/EDX microscope.

Fillers F content migrated from particles were determined by Particle Size data Analysis, using a Malvern Mastersizer 2000 laser granulometer and ethanol as dispensing medium.

### EXAMPLE 1

Blends were made according to TABLE 1.

Trial compositions were produced using granules of PHB+30% of filler F (FIR) previously prepared using a twin screw extruder SHJ20. The granules of PHB with 30 wt % of FIR additives were obtained by melt extruding process mixing 69 wt % of PHB with 1.0 wt % of citric acid, 15.75 wt % of tourmaline, 10.5 wt % of barium sulphate and 3.75 wt % of titanium dioxide. The extruder temperature profile of the various zones of the extruder during the process varied from 173° C. to 151° C. and the rotation speed were 65 rpm.

Then, the granules were introduced with agent C and PEG in a twin-screw extruder device rotating at 300 rpm to prepare the melt blend.

The introduction was carried out using feeding by weight. The agent C in liquid phase was mixed with PHB previously. PHB and PEG were in solid form, granules and pellets, respectively.

During the first stage, an adequate screw profile is needed to promote an efficient blending of the material. After a profile of screw and temperature was applied according to the nature of the products and a residential time enough to provide the rupture of droplets formed from HIPE emulsion.

The extruder conditions used during the process were: rotation of 300 rpm, temperatures of the various zones of the extrusion screw between 166 and 170° C. and the throughput of 0.4 kg/h.

The melt blends were cooled into water, and the solubilization of the PEG from the blend occurs instantaneously for the most trial compositions.

TABLE 1
Trial composition (wt %)

| | | | PEG 20000 | |
|---|---|---|---|---|
| Trial | PHB + FIR | PEG 6000 Da | Da | Agent C |
| 1 | 20 | 80 | — | 0 |
| 2 | 25 | 65 | — | 10 |
| 3 | 45 | 45 | — | 10 |
| 5 | 25 | — | 65 | 10 |
| 6 | 45 | — | 45 | 10 |
| 8 | 45 | — | 50 | 5 |

The final particles were recovered by centrifugation and dried at 100° C. overnight.

Trial 1 did not disaggregate instantly when the cooled blend was introduced into water. This trial resulted in thermoplastic polymer M being the continuous phase, and thus no spherical particles were obtained for this trial.

### EXAMPLE 2

Particle size distribution for the trial compositions of example 1 was analysed and the results were presented in TABLE 2.

The particle size distribution of the samples was determined using a Malvern Mastersizer 3000 laser granulometer coupled with the Hydro LV accessory, which allows analysis under solvent dispersion. Mastersizer 3000 uses laser diffraction to measure particle size and particle size distribution of materials. It measures the intensity of the scattered light as the laser beam interacts with the dispersed particles of the sample.

The trial compositions were analysed immediately after their addition to the granulometer using ethanol as dispersing medium.

TABLE 2
Particle size distribution

| Trial | d(0.1) (μm) | d(0.5) (μm) | d(0.9) (μm) |
|---|---|---|---|
| 1 | 270 | 430 | 675 |
| 2 | 0.91 | 45.5 | 106 |
| 3 | 0.82 | 31.9 | 134 |
| 5 | 0.85 | 6.0 | 30.4 |
| 6 | 0.86 | 9.8 | 37.2 |
| 8 | 0.86 | 8.9 | 28.6 |

The results found for trial compositions analysed for particle size distribution showed a D50 in the range of 6 μm to 50 μm.

### EXAMPLE 3

Scanning Electron Microscopy:

The procedure for assessing sphericity of the spherical polymeric particles was carried out by Scanning Electron Microscopy (SEM) using the major and minor axes passing through the center of particle. Each particle identified in the Scanning Electron Microscopy (SEM) was collected, and the axes were determined perpendicular to each other, and the spherical shape factor was calculated as the ratio of minor axes to major axes. At least 100 determinations (50 particles) were performed for each assay.

The results were described at TABLE 3 for the trial compositions of example 1.

TABLE 3
Spherical Shape factor

| Trial | Shape factor |
|---|---|
| 1 | 0.30 |
| 2 | 0.84 |
| 3 | 0.76 |
| 5 | 0.91 |
| 6 | 0.89 |
| 8 | 0.89 |

As can be seen in TABLE 3, by adding the agent C during the extrusion mixing step, shape factors higher than 0.75 were observed, resulting in spherical particles. When no agent C is added during the extrusion mixing step, spherical particles were not obtained.

### EXAMPLE 4

Migrated Fillers F from Particles:

The fillers F migrated from thermoplastic polymeric matrix M content were determined by the particle size distribution data according to the volume difference between total particles and free fillers F and calculated using mass ratio between fillers F and total particles. Same density was assumed for all particles and using the volume of particles having diameter smaller than 1.5 μm, represented by free fillers F, the mass was calculated.

The results obtained were described at TABLE 4 for the trial compositions of example 1

TABLE 4
Migrated fillers F from particle.

| | Migrated fillers F |
|---|---|
| Trial | (mg/kg particles PHB + FIR) |
| 1 | 140000 |
| 2 | 31 |
| 3 | 23 |
| 5 | 890 |
| 6 | 630 |
| 8 | 2260 |

The migrated fillers F parameter found for the spherical particles when agent C were added varied from 23 to 2260 mg/kg, while when no agent C were added, the migrated fillers F reached 140000 mg/kg.

### EXAMPLE 5

Blends were obtained according to TABLE 5 using manufacture process according to example 1.

Trial compositions were produced using granules of PA 6.6+30% of fillers F (FIR) previously prepared using a twin screw extruder SHJ20. The granules of PA 6.6 with 30 wt % of FIR additives were obtained by melt extruding process mixing 70 wt % of PA 6.6 with 15.75 wt % of tourmaline, 10.5 wt % of barium sulphate and 3.75 wt % of titanium dioxide. The extruder temperature profile of the various zones of the extruder during the process varied from 265° C. to 284° C. and the rotation speed were 460 rpm.

Then, the granules were introduced with agent C and PEG in a twin-screw extruder device, the temperature profile of the various zones during the process varied from 250° C. to 270° C. rotating at 300 rpm to prepare the melt blend.

The introduction was carried out using feeding by weight. The agent C in liquid phase was mixed with PA 6.6+FIR previously. PA 6.6+FIR and PEG were in solid form, granules and pellets, respectively.

During the first stage, an adequate screw profile is needed to promote an efficient blending of the material. After, a profile of screw and temperature was applied according to the nature of the product and a residential time enough to provide the rupture of droplets formed from HIPE emulsion.

The extruder conditions used during the process were: rotation of 300 rpm, temperatures of the various zones of the extrusion screw between 250 and 270° C. and the throughput of 0.4 kg/h.

The melt blends were cooled into water, and the solubilization of the PEG from the blend occurs instantaneously for the most trial compositions.

TABLE 5
Trial composition (wt %)

| | PA 6.6 + | PEG 35000 | |
|---|---|---|---|
| Trial | FIR | Da | Agent C |
| 1 | 30 | 60 | 10 |
| 2 | 45 | 45 | 10 |
| 3 | 50 | 45 | 5 |

The final particles were recovered by centrifugation and dried at 100° C. overnight.

### EXAMPLE 6

Particle size distribution for the trial compositions of example 5 was analysed and the results were presented in TABLE 6.

The particle size distribution of the samples was determined using a Malvern Mastersizer 3000 laser granulometer coupled with the Hydro LV accessory, which allows analysis under solvent dispersion. Mastersizer 3000 uses laser diffraction to measure particle size and particle size distribution of materials. It measures the intensity of the scattered light as the laser beam interacts with the dispersed particles of the sample.

TABLE 6
Particle size distribution

| Trial | d(0.1) (μm) | d(0.5) (μm) | d(0.9) (μm) |
|---|---|---|---|
| 1 | 4.67 | 25.4 | 78.9 |
| 2 | 2.95 | 28.3 | 122 |
| 3 | 9.14 | 37.8 | 91.8 |

The results found for trial compositions analysed for particle size distribution showed a D50 in the range of 20 μm to 40 μm.

### EXAMPLE 7

Scanning Electron Microscopy:

The procedure for assessing sphericity of the spherical polymeric particles was carried out by Scanning Electron Microscopy (SEM) using the major and minor axes passing through the center of particle. Each particle identified in the Scanning Electron Microscopy (SEM) was collected, and the axes were determined perpendicular to each other, and the spherical shape factor was calculated as the ratio of minor axes to major axes. At least 100 determinations (50 particles) were performed for each trial.

The results were described at TABLE 7 for trial compositions of example 5.

TABLE 7
Spherical Shape factor

| Trial | Shape factor of PA66 + FIR particles |
|---|---|
| 1 | 0.98 |
| 2 | 0.98 |
| 3 | 0.98 |

As can be seen in TABLE 7, shape factors of PA 6.6+FIR particles using agent C resulting in spherical particles.

### EXAMPLE 8

Migrated Fillers F from Particles:

The fillers F migrated from thermoplastic polymeric matrix M content were determined by the particle size distribution data according to the volume difference between total particles and free fillers F and calculated using mass ratio between fillers F and total particles. Same density was assumed for all particles and using the volume of particles having diameter smaller than 1.5 μm, represented by free fillers F, the mass was calculated.

The results obtained were described at TABLE 8 for the trial compositions of example 5.

TABLE 8
Migrated fillers F from particles.

| | Migrated fillers F |
|---|---|
| Trial | (mg/kg particles PA 6.6 + FIR) |
| 1 | 12.0 |
| 2 | 16.0 |
| 3 | 1.0 |

The migration of fillers F is less than 1% which means did not have a significant value.

Therefore, surprisingly, it has been found a process, using the same agent C and different types of polymers, able to produce spherical polymeric particles in a shape and size controlled way, containing fillers F dispersed in the polymeric matrix wherein said process was able to guarantee the permanence of the fillers F inside the thermoplastic polymeric matrix M during co-extrusion process.

It should be understood that the invention is not limited by the above description but rather by the claims appended hereto.

1. A process for preparing spherical particles comprising a thermoplastic polymer matrix (M) comprising at least two fillers (F), dispersed in the thermoplastic polymeric matrix (M), which comprises the following steps:
A—melt-blending a mixture comprising:
a) at least one thermoplastic polymer matrix (M) comprising up to 50% of the at least two fillers (F) dispersed therein;
b) at least one compound (P), different from the at least one thermoplastic polymer matrix (M), not miscible with the at least one thermoplastic polymer matrix (M) and selected in the group consisting in polyglycols, polysaccharides, polyolefins, polyvinyl alcohols, silicones, waxes, and mixtures thereof; and
c) at least one agent (C) which is an amphiphilic compound having a first part of its structure that can react chemically or physically with the thermoplastic polymer matrix (M) and a second part of its structure that can react chemically or physically with the at least one compound (P), and in which the first part of its structure does not contain a polymer chain identical to the thermoplastic polymer matrix (M);
thus forming an emulsion containing a continuous phase of the at least one compound (P) and the at least one agent (C) and droplets of the thermoplastic polymer matrix (M) and the at least two fillers (F);
B—cooling the melt blend obtained at step A at a temperature below the softening temperature of the melt blend to form a cooled blend,
C—putting the cooled blend into a solvent wherein the at least one compound (P) and the at least one agent (C) are soluble to provide solubilization of the at least one compound (P) and the at least one agent (C),
D—recovering the spherical particles comprising the thermoplastic polymer matrix (M) and the at least two fillers (F) dispersed therein.

2. The process according to claim 1, wherein the thermoplastic polymer matrix (M) is selected from a synthetic polymer or a biodegradable polymer.

3. The process according to claim 2, wherein the synthetic polymer is selected from at least one member of the group of polyesters, polyolefins, polymers based on a cellulose ester, acrylic polymers and copolymers, polyamides, copolymers in any proportions of these polymers, and mixtures thereof.

4. (canceled)

5. The process according to claim 2, wherein the thermoplastic polymer matrix M is a biodegradable polymer and is selected from at least one member of the group consisting of:
polylactic acid (PLA),
polylactic-co-glycolic acid (PLGA),
polyhydroxyalkanoates (PHAs),
thermoplastic starches (TPS),
poly(butylene Succinate) (PBS),
poly(butylene Succinate adipate) (PBSA),
polybutylene adipate (PBA),
polybutylene adipate terephthalate (PB AT) or Polylactic acid (PLA)/polycaprolactone (PCL), and
a thermoplastic polymer comprising additive(s) that provide(s) a biodegradable property.

6. The process according to claim 5, wherein the thermoplastic polymer matrix (M) is a polyhydroxyalkanoate (PHA).

7. The process according to claim 1, wherein the at least two fillers (F) comprises at least two mineral fillers having properties of absorption and/or emission in the far infrared region ranging from 2 μm to 20 μm, wherein the at least two mineral fillers (F) are selected from the group consisting of oxides, sulfates, carbonates, phosphates and silicates.

8. (canceled)

9. (canceled)

10. The process according to claim 7, wherein one of the at least two mineral fillers is a silicate, selected from the group consisting of actinolite, micas, tourmaline, serpentine, kaolin, montmorillonite, zeolite and mixtures thereof.

11. The process according to claim 1, wherein the weight proportion of the at least two fillers (F) relative to the total weight of the spherical particles is greater than or equal to 1%.

12. The process according to claim 1, wherein the weight proportion of the at least two fillers (F) relative to the total weight of the spherical particles is less than or equal to 50%.

13. The process according to claim 1, wherein the at least one compound (P) is selected in the group consisting of polyoxyethylenes (POE) and polyalkylene glycols (PAG).

14. The process according to claim 1, wherein the at least one compound (P) is a polyethylene glycol with a molecular weight ranging from 1500 to 60000 g/mol.

15. The process according to claim 1, wherein the at least one agent (C) is an ethoxylated/propoxylated block polymer with a molecular weight ranging from 500 to 10000 g/mol., and with a PO/EO ratio ranging from 2 to 10.

16. (canceled)

17. The process according to claim 1, wherein the solvent of step C is selected in the group consisting of water, methanol, ethanol, isopropanol and butanol.

18. The process according to claim 1, wherein the spherical particles are dried after step D.

19. The process according to claim 1, wherein the melt blend of step A comprises:
a) from 15 to 80 wt. % of the at least one thermoplastic polymer matrix (M) comprising the at least two fillers dispersed therein, which are PHB or PA 6.6 comprising three fillers being titanium dioxide, barium sulphate and tourmaline, from 20 to 50 wt. %, and;
b) from 15 to 80 wt. % of the at least one compound (P) being a PEG;
c) from 1 to 20 wt. % of the at least one agent (C) being an ethoxylated/propoxylated block copolymer.

20. The process according to claim 1, wherein step A takes place at a temperature above 100° C. and below 300° C.

21. The process according to claim 1, in which the melt blend is processed by extrusion in an extruder selected from endless screw mixers or stirrer mixers.

22. The process according to claim 1, wherein the spherical particles contain migrated fillers in an amount not more than 5000 mg/kg.

23. Spherical particles of polymer obtained by the process of claim 1, wherein the average particle size size D50 is ranging from 5 μm to 60 μm.

24. (canceled)

25. The spherical particles according to claim 23, having a spherical shape factor ratio being selected from 0.5 to 1.0.

26. (canceled)
